(19) United States
(12) Patent Application Publication
(10) Pub. No.: US 2009/0198171 A1
(43) Pub. Date: Aug. 6, 2009

(54) DEVICE FOR USE IN AN ARRANGEMENT FOR MONITORING AN ACCESS TO A PATIENT, AND METHOD FOR MONITORING A PATIENT ACCESS, IN A PARTICULAR VASCULAR ACCESS IN EXTRACORPOREAL BLOOD TREATMENT

(76) Inventors: Wolfgang Kleinekofort, Kelkheim (DE); Jörg Jonas, Wehrheim (DE)

Correspondence Address: KENYON & KENYON LLP, ONE BROADWAY, NEW YORK, NY, 10004, US

(21) Appl. No.: 12/304,181

(22) PCT Filed: Jun. 6, 2007
(86) PCT No.: PCT/EP2007/004994

§ 371 Date: Dec. 10, 2008

(30) Foreign Application Priority Data
Jun. 10, 2006 (DE) 10 2006 027 054.1

(51) Int. Cl. A61M 39/10

(52) U.S. Cl. 604/6.16

(57) ABSTRACT

The present invention relates to a device for monitoring a patient access, in particular a vascular access in extracorporeal blood treatment. The device is used to establish a substantially parallel connection between the arterial and venous tubes, or the arterial and venous puncture needles. If the venous needle slips out of the vascular access, the arterial cannula is forcibly withdrawn. This incident is reliably detected by the known protection systems which monitor the pressure in the arterial tube and/or an air intake. In addition, the present invention relates to an extracorporeal blood treatment device and to a method for monitoring a patient access, wherein the venous and arterial tubes or the puncture needles are connected to each other.

### CROSS REFERENCE TO RELATED APPLICATIONS

This is a 371 national phase application of PCT/EP2007/004994 filed Jun. 6, 2007, claiming priority to German Patent Application No. 10 2006 027 054.1 filed Jun. 10, 2006.

### FIELD OF INVENTION

The present invention relates to a device for an arrangement for monitoring an access to a patient, whereby a fluid is removed from the patient with a first flexible-tube line, which has a first patient connection, and the fluid is supplied to the patient via a second flexible-tube line, which has a second patient connection. In particular, the present invention relates to an arrangement for monitoring a vascular access in an extracorporeal blood treatment. Moreover, the present invention relates to a blood treatment apparatus with an extracorporeal blood circuit, which comprises an arterial flexible-tube line with an arterial puncture cannula and a venous flexible-tube line with a venous puncture cannula, and an arrangement for monitoring the vascular access. Furthermore, the present invention relates to a method for monitoring an access to a patient, in particular for monitoring the vascular access in an extracorporeal blood treatment.

### BACKGROUND OF THE INVENTION

In the field of medical technology, a large number of apparatuses are kmown for removing fluids from a patient or delivering fluids to a patient via a flexible-tube line. The access to the patient generally takes place with a catheter for introduction into body organs, or a cannula for puncturing vessels. Correct access to the patient must be ensured during the examination or treatment. It is therefore necessary to monitor the patient access.

In methods for chronic blood cleaning therapy such as hemodialysis, hemofiltration and hemodiafiltration, blood is conveyed via an extracorporeal circuit with a maximum flow of 600 ml/min. Arteriovenous fistulas, vascular implants or catheters are used to access to the patient's vascular system. Typical flows within the vascular access are about 1100 ml/min. The connection of the patient to the extracorporeal circuit takes place via dialysis cannulas, with which the fistula or the vascular implant is punctured.

If, during the treatment, the patient connection between the extracorporeal circuit and the vascular system becomes detached or a blood leakage occurs in the extracorporeal circuit, the patient can be prevented from bleeding to death only if the extracorporeal blood flow is stopped within a few seconds. Extracorporeal blood circuits are therefore equipped with protection systems which permanently monitor the arterial and venous pressure (P_{art.} and P_{ven.}) within the system as well as the intake of air into the extracorporeal circuit. In the event of an alarm, the blood pump is stopped, the venous clamp is closed, and an acoustic or optical warning signal is emitted.

The known hemodialysis apparatuses generally have an arrangement for monitoring the vascular access that is based on a measurement of the arterial or venous pressure in the extracorporeal circuit. The known protection systems generally respond when the arterial or venous pressure changes by more than ±60 torr. The alarm limits are selected so that a change in position by the patient does not trigger an alarm.

If the connection between the patient and the dialysis apparatus becomes detached at the arterial connection, i.e. at the cannula which produces the blood flow from the patient to the extracorporeal circuit, the pressure-based protection system on the machine side responds rapidly, as will be explained below. The arterial puncture cannula forms the greatest flow resistance in the extracorporeal circuit. If air is sucked via the cannula into the arterial underpressure system of the extracorporeal circuit, the arterial pressure in the extracorporeal circuit abruptly collapses. As a result of the resulting arterial pressure alarm, the blood pump is stopped and the venous clamp closed, so that the possibility of the patient bleeding to death is eliminated.

However, in the case where the venous puncture cannula becomes detached from the vascular access, the response of the pressure-based protection system is not guaranteed. The cleaned blood is fed at excess pressure to the patient on the venous side, whereby the venous excess pressure is proportional to the delivery quantity of the blood pump and to the flow resistance of the venous branch of the extracorporeal circuit. A penetration of air through the cannula into the extracorporeal circuit—as would be the case on the arterial underpressure side—is thus ruled out. The flow resistance of the venous cannula does not therefore change and the venous pressure on the machine side only falls by the amount of the pressure in the patient's vascular access. With intact fistulas, the arterial pressure in the vascular access amounts to approx. 30 torr and with intact vascular implants (PTFE graft) to approx. 50 torr. The venous pressure lies at approx. 25 torr (fistula) or at approx. 40 torr (graft). Therefore, the venous pressure change in the extracorporeal circuit is too small to trigger a response of the pressure-based protection system. Only in the case where the venous cannula lies well beneath the fistula after slipping out of the vascular access can the additional hydrostatic pressure difference between the venous pressure sensor and the cannula trigger a machine alarm. However, in known devices, no alarm is triggered, and the patient bleeds to death in an extremely short time.

A method for the monitoring of a vascular access in an extracorporeal blood treatment is known from US 2004/0186415 A1 (WO 2004/084972 A2). US 2004/0186415 A1 proposes combining the venous and arterial puncture needle into a double-lumen cannula, wherein the arterial cannula surrounds the venous cannula concentrically. The two cannulas end in a common connection piece for the arterial and venous blood line. This is intended to combine the advantages of so-called single-needle dialysis in terms of patient safety with the advantages of the continuous blood flow of double-needle dialysis. Drawbacks of such a double-lumen cannula in combination with the proposed connection piece are the size of the puncture point on the patient and the high recirculation rates during the blood treatment, because the arterial inlet and the venous outlet are only a few millimetres apart from one another due to the design. In addition, double-lumen cannulas have not hitherto managed to establish themselves in the market on account of their high cost. Furthermore, the use of a double-lumen cannula limits the possible treatment methods.

Since, with the described double-lumen cannula, the slipping-out of the venous cannula automatically leads to the detachment of the arterial cannula, the venous patient access can be monitored by the fact that the arterial patient access is monitored by the air detection systems that are present as standard. The known system cannot however be used for a dialysis treatment with separate arterial and venous needles, i.e. with two separate puncture points, which is preferably used on account of the higher blood flows.

WO 02/072179 A1 proposes a different method than US 2004/0186415 A1 for solving the problem of incorrect patient accesses. WO 02/072179 A1 proposes joining the arterial and venous puncture cannula with a joining element, which is designed in such a way that the two cannulas point in opposite directions. The cannulas should preferably enclose an angle of at least 30°, in particular at least 45°. The cannulas are thus intended to get a better hold, and forcible tearing-out, for example in the case of confused patients, is thus intended to be prevented. There is however the risk of one of the cannulas being pushed into the puncture point in an uncontrolled manner, causing pain to the patient or even damaging or piercing the opposite vascular wall.

### SUMMARY OF THE INVENTION

The problem underlying the present invention is to provide a device which makes it possible, without major outlay, to increase the reliability of the arrangements for monitoring an access to a patient. Moreover, the problem of the present invention is to provide a blood treatment apparatus with an arrangement for monitoring a patient access with increased reliability. Furthermore, a problem of the present invention is to provide a method with which the reliability of the arrangements for monitoring a patient access, which includes both a first and a second patient connection, can be increased.

The device according to the present invention, with which the reliability of the known arrangements for monitoring a patient access can be increased, has means for the separable joining of the first patient connection or the first flexible-tube line with the second patient connection or the second flexible-tube line. The joining means are designed in such a way that the patient connections or flexible-tube lines can be joined to one another separably and in an essentially parallel orientation. The patient connections, in particular the puncture cannulas in the extracorporeal blood treatment, do not point in different directions. For this purpose, either the patient connections themselves, or the flexible-tube lines, can be joined to one another. It is however also possible to join the first patient connection with the second flexible-tube line or the second patient connection with the first flexible-tube line. In any event, the joining of the flexible-tube lines should take place in the vicinity of the puncture cannulas in order to keep the cannulas in a parallel orientation.

The joining means can be designed differently, however the joining must be separable, so that the puncture cannulas can first be inserted and then the puncture cannulas or flexible-tube lines can be joined to one another. The fact that the joining is separable is also of advantage for correcting the seating of the puncture cannulas.

Since the puncture cannulas or flexible-tube lines are joined, the slipping-out of the venous puncture cannula automatically leads to the detachment of the arterial puncture cannula. Since the puncture cannulas are orientated essentially parallel to one another, the arterial puncture cannula can easily slip out if the venous puncture cannula slips out, but without being able to be pushed into the puncture point or even being able to damage or pierce the opposite wall of the blood vessel. It is thus ensured that, in the event of the venous puncture cannula being pulled out, the arrangement for monitoring the patient access which monitors the correct seating of the arterial puncture cannula responds. In this case, air is sucked in via the arterial line, which can be reliably detected with the standard monitoring arrangement according to the known method. For example, a standard arterial pressure alarm device can respond. A standard air detector can also detect an air intake into the arterial line. Irrespective of this, the blood treatment apparatus will always trigger an alarm even in the event of a malfunction of the arterial pressure measurement, because the pulling-out of the puncture cannula leads to an alarm being triggered by a standard level control device on the drip chamber.

In the event of slipping-out of the puncture cannula, the arterial pressure measurement will respond to the sudden pressure change, the blood pump will be stopped, and the venous flexible-tube clamp will be closed due to the resulting pressure alarm. With the device according to the present invention, the air sucked into the arterial line can be detected with the standard air detector and the blood pump stopped and the venous clamp closed due to the resulting alarm.

Irrespective of the triggering of the alarm, there is with the device according to the invention no risk of the patient bleeding to death in the event of a disconnection, because both the venous and the arterial cannula are separated from the patient, so that the patient is uncoupled from the extracorporeal blood circuit. The feared pumping the patient empty of blood can thus be eliminated.

In principle, it would be possible to design the device according to the present invention in such a way that the arterial cannula is first withdrawn before the venous cannula is fully withdrawn. This has the advantage that an alarm is emitted before the patient's blood is lost. Pulling-out of the arterial cannula before the venous cannula is preferably achieved by the fact that a flexible-tube segment of the venous flexible-tube line between the venous cannula and the joining element according to the invention is designed in the form of a loop, so that the loop can tighten when the venous flexible-tube line is subjected to a tractive load. The loop thus first tightens in the event of a tractive load, as a result of which the tractive force is prevented from being transferred immediately to the cannula. For this purpose, the device according to the invention can have fixing means, with which the flexible-tube line segment can be put into a loop. The fixing means can be designed, for example, as a clip or the like as described in WO 2007/104350.

In a preferred embodiment of the device according to the present invention, the joining means comprises a first attachment element for the attachment to the first patient connection or the first flexible-tube line and a second attachment element for the attachment to the second patient connection or the second flexible-tube line.

In a first embodiment, at least one of the two attachment elements is designed in such a way that the attachment element can be attached separably to the patient connection or the flexible-tube line. The at least one attachment element is preferably designed in such a way that the attachment element can be fixed in a clamped fashion to the flexible-tube line. For example, the attachment element can be designed as a clip or the like. Both attachment elements can of course also be designed for the preferably clamped fixing. As a result, it is possible for the flexible-tube lines to be joined easily to one another and separated again. The attachment means can be provided as a single-use article intended for on-off use or also for repeated use.

In a second embodiment, the attachment means are designed in such a way that the attachment elements can be joined to one another separably. With this alternative embodiment, the attachment elements can first be firmly joined with the patient connection or the flexible-tube line. The attachment elements can then preferably be joined to one another separably with a snap-in fastener, whereby the snap-in fastener is preferably designed as a press-stud. Any other kind of joining is however also possible as long as the essentially parallel orientation of the puncture cannulas is ensured.

In a further embodiment, the patient connections each comprise a puncture cannula and two wings projecting laterally from the cannula, which are joined to the puncture cannula so as to be rotatable with the cannula about its axis. Such patient connections are known as puncture wings. A particularly preferred embodiment makes provision, with the known puncture wings, to join the free ends of the wings to one another separably. The wings are thereby designed in such a way that the wings can be folded together to form an eyelet, within which a flexible-tube line can be fixed in a clamped manner. The free ends of the wings can preferably be joined to one another with a snap-in fastener, which is preferably designed as a press-stud.

This embodiment has the advantage that the joining means is a one-piece component of the two puncture wings. Consequently, the joining means cannot get lost, but is as it were made available with the puncture wing. However, the drawback lies in the fact that a subsequent joining of conventional patient connections is not possible.

In the blood treatment apparatus according to the present invention, which has an extracorporeal blood circuit, the arterial flexible-tube line or the arterial puncture cannula is joined separably with the venous flexible-tube line or the venous puncture cannula, so that the arterial puncture cannula is automatically pulled out in the event of the venous puncture cannula slipping out.

In principle, the two cannulas can be joined to one another separably with adhesive tape or the like. A preferred embodiment of the invention, however, provides for joining by means of the device according to the invention.

The method according to the present invention provides for joining of the patient connections or flexible-tube lines in order that, if one patient connection becomes detached, the other patient connection is automatically pulled out.

### BRIEF DESCRIPTION OF THE DRAWINGS

Various examples of embodiment of the invention are explained below in greater detail by reference to the drawings.

FIG. 1 shows the main components of a hemodialysis apparatus together with a device for monitoring the patient access in a greatly simplified diagrammatic representation.

FIG. 2 shows a first embodiment of the device according to the invention in perspective view.

FIG. 3 shows a second embodiment of the device according to the invention in perspective view.

FIGS. 4a and 4b show a further embodiment of the device according to the invention.

FIG. 5 shows a cross-sectional view along line V-V of FIG. 4b.

### DETAILED DESCRIPTION OF THE DRAWINGS

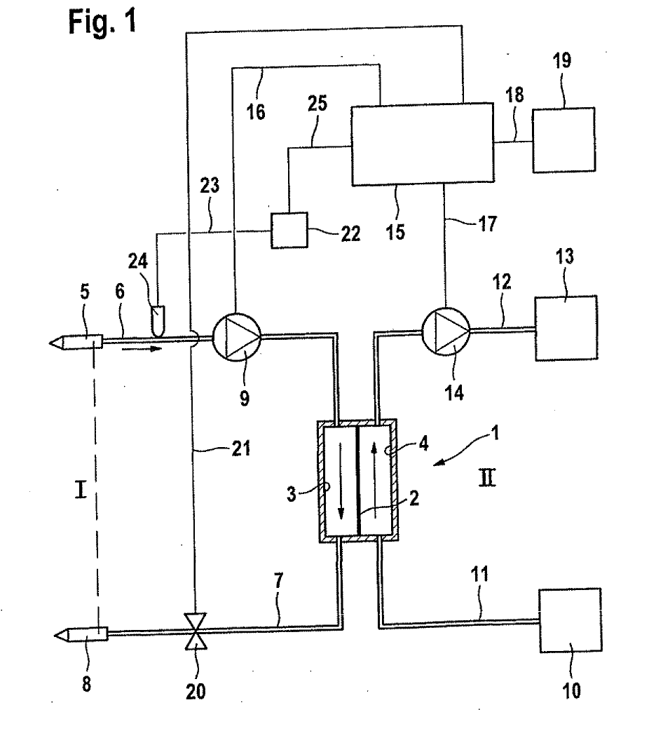

FIG. 1 shows the main components of a hemodialysis apparatus, which has an arrangement for monitoring the arterial and venous vascular access. The hemodialysis apparatus has a dialyser 1, which is divided by a semi-permeable membrane 2 into a blood chamber 3 and a dialysing fluid chamber 4. Connected to one of the patient's arteries by means of an arterial puncture cannula 5 is an arterial flexible-tube line 6, which leads to the inlet of blood chamber 3 of the dialyser. Leading away from the outlet of blood chamber 3 of the dialyser is a venous flexible-tube line 7, which is connected by means of a venous puncture cannula 8 to one of the patient's veins. Arterial flexible-tube line 6 is inserted into an occluding blood pump 9, which conveys the blood in extracorporeal blood circuit I.

Dialysing fluid circuit II of the hemodialysis apparatus comprises a dialysing fluid source 10, to which a dialysing fluid supply line 11 is connected, which leads to the inlet of dialysing fluid chamber 4 of the dialyser. Leading away from the outlet of dialysing fluid chamber 4 of dialyser 1 is a dialysing fluid discharge line 12, which leads to a drain 13. A dialysing fluid pump 14 is incorporated into dialysing fluid discharge line 12.

The control of the dialysis apparatus is assumed by a central control unit 15, which controls blood and dialysing-fluid pumps 9, 14 via control lines 16, 17, respectively. Central control unit 15 is connected via a data line 18 to an alarm unit 19, which in the event of a malfunction emits an optical and/or acoustic alarm.

Located downstream of blood chamber 3 of dialyser 1 on venous flexible-tube line 7 is an electromagnetically operated flexible-tube clamp 20, which is closed via a further control line 21 by central control unit 15 if the venous puncture cannula (needle) slips out of the vascular access. Furthermore, control unit 15 stops blood pump 9 after the slipping out of the cannula.

In order to monitor the arterial and venous vascular access, the dialysis apparatus has a monitoring arrangement 22, which is connected via a data line 23 to a pressure sensor 24 and/or air detector, that measures the pressure in arterial flexible-tube line 7 and/or detects an air intake into the flexible-tube line. Monitoring arrangement 22 communicates with central control unit 15 via a further data line 25.

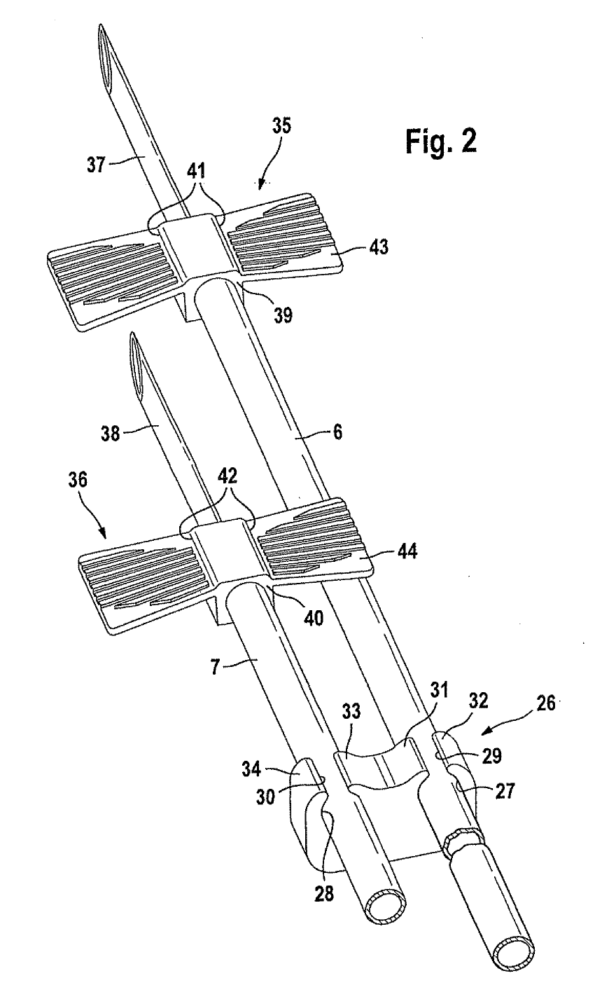

FIG. 2 shows a first embodiment of the device according to the invention for the connection of the flexible-tube lines of the dialysis apparatus described by reference to FIG. 1, together with arterial and venous flexible-tube lines 6, 7 and arterial and venous puncture cannulas 5, 8.

The device according to the invention is a joining piece 26, with which arterial flexible-tube line 6 can be joined separably with venous flexible-tube line 7. The joining piece 26 is designed in the manner of a clip fastener with a symmetrical structure. Joining piece 26 is an injection molded part made of plastic with two cylindrical cut-outs 27, 28, with parallel axes. Cut-outs 27, 28 have a diameter which is slightly smaller than the diameter of flexible-tube lines 6, 7, so that the flexible-tube lines are joined in a clamped manner by the joining piece 26.

Joining piece 26 has at the upper side two slits 29, 30, through which flexible-tube lines 6, 7 can be pushed into cut-outs 27, 28 or pulled out. The distance between inner legs 31, 33 and outer legs 32, 34 is dimensioned in such a way that the flexible-tube lines are compressed when they are being pushed in or pulled out.

The embodiment of FIG. 2 provides known puncture wings 35, 36 as patient accesses. Arterial puncture wing 35 and venous puncture wing 36 each have an arterial and venous puncture cannula 37 and 38 respectively, which are joined firmly by an essentially cylindrical joining piece 39, 40 to arterial flexible-tube line 6 or venous flexible-tube line 7 respectively. Formed onto cylindrical joining pieces 39, 40, in each case by means of film hinges 41, 42, are wings 43, 44 which project laterally from the joining pieces. Wings 43, 44 are rotated upwards for the insertion of puncture cannulas 37, 38, so that the free ends of the wings lie next to one another with their inner sides. The wings form a grip part, which is held with thumb and forefinger when the cannulas are inserted.

The flexible-tube lines are joined to one another by joining piece 26 for the insertion of the cannulas. Cannulas 37, 38 are first inserted. The flexible-tube lines lying parallel to one another are then joined to one another by joining piece 26 according to the invention, the flexible-tube lines being inserted into the joining piece, so that the flexible-tube lines and the cannulas are oriented parallel to one another.

If venous cannula 38 should slip out, for example due to an unintentional tug on venous flexible-tube line 7, arterial cannula 37 is automatically also pulled out. In this case, air is sucked via the arterial cannula into arterial flexible-tube line 6, so that the arterial underpressure in the extracorporeal circuit abruptly collapses. The arterial pressure, which is measured with arterial pressure sensor 24, then falls below a preset limiting value, which is detected by pressure monitoring arrangement 22. The pressure monitoring arrangement can however also have an air detector which detects an air intake. Pressure monitoring arrangement 22, which communicates with control unit 15, then initiates an acoustic and/or optical alarm which is emitted by alarm unit 19.

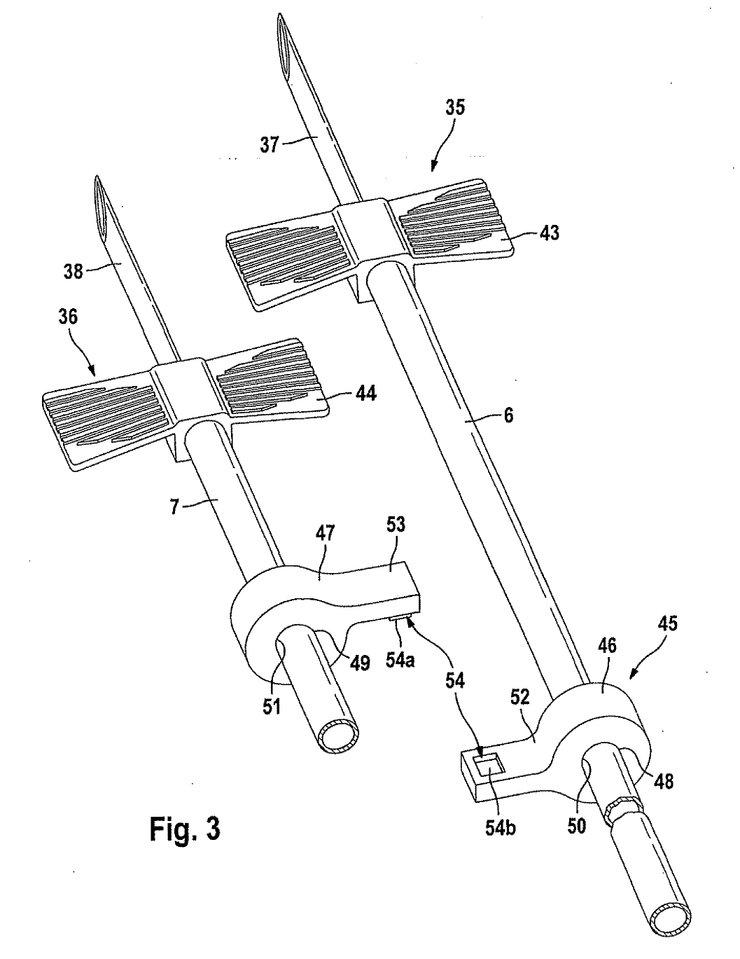

FIG. 3 shows a second embodiment of the joining means according to the invention, together with flexible-tube lines 6, 7 and puncture wings 35, 36, whereby the same reference numbers are used for the same parts.

Joining means 45 differs from joining piece 26 by the two-part structure. Joining means 45 comprises an arterial joining part 46 and a venous joining part 47, which are firmly joined with arterial and venous flexible-tube lines 6, 7. Both joining parts 46, 47 have a portion 48, 49 surrounding respective flexible-tube lines 6, 7, whereby flexible-tube lines 6, 7 sit firmly in corresponding holes 50, 51 of these portions. Portions 48, 49, which surround the flexible-tube lines, are joined to portions 52, 53, which taper to a flat section and are provided with a snap-in fastener 54. Snap-in fastener 54 is designed in the manner of a press-stud. Press-stud 54 comprises a rectangular projection 54a at the lower side of inwardly pointing portion 53 of venous joining part 47 and a rectangular cut-out 54b at the upper side of inwardly pointing portion 52 of arterial joining part 46. Projection 54a and cut-out 54b are designed in the manner of a press-stud, in such a way that the projection can be pushed in a snap-in fashion into the cut-out, so that the two joining parts are joined to one another in a torsionally resistant manner with a parallel orientation of flexible-tube lines 6, 7 and cannulas 37, 38. With this embodiment, slipping-out of the venous cannula again automatically leads to pulling-out of the arterial cannula.

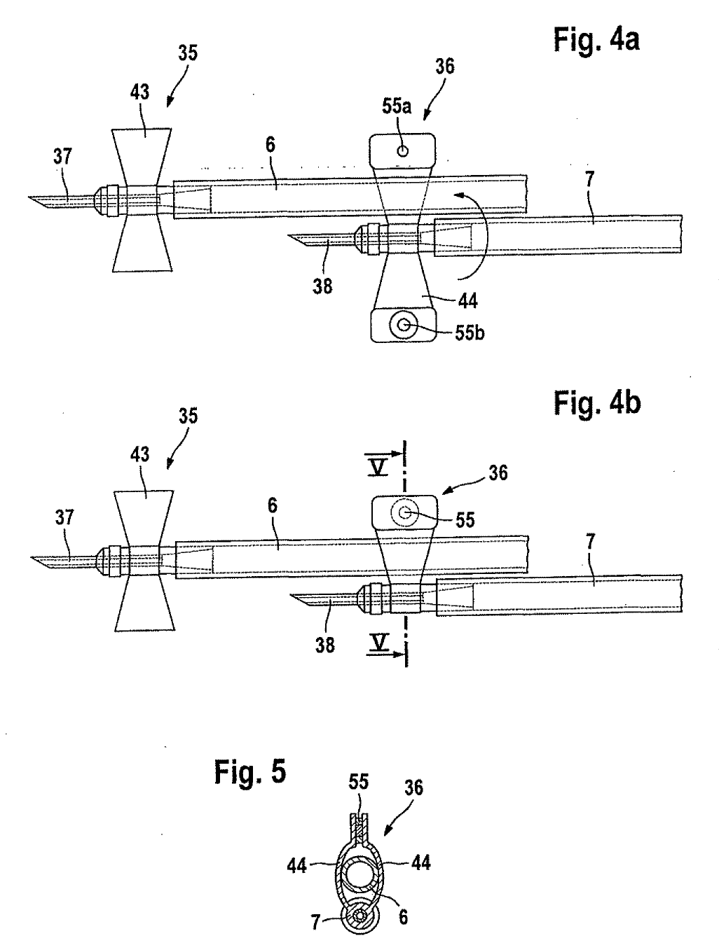

FIGS. 4a and 4b show a further embodiment of the invention. This embodiment differs from the embodiments described by reference to FIGS. 2 and 3 in that it is not flexible-tube lines 6, 7 that are joined to one another, but rather puncture wing 36 of the venous patient connection that is joined with arterial flexible-tube line 6. It is however also possible to join the arterial puncture wing with the venous flexible-tube line. The same parts are again provided with the same reference numbers.

Wings 44 of venous puncture cannula 36 are designed in such a way that the wings can be folded together upwards with a clamped fixing of arterial flexible-tube line 6. FIG. 4a shows venous puncture wing 36 before its wings 44 are folded together, whilst FIG. 4b shows puncture wing 36 which fixes arterial flexible-tube line 6 in a clamped fashion. Provided at the free ends of wings 44 is a snap-in fastener 55, with which the wings can be joined to one another. Snap-in fastener 55 is again designed in the manner of a press-stud, which has a circular projection 55a at the inner side of one wing 44, which is pushed in a snap-in fashion into a circular cut-out 55b at the opposite inner side of the other wing.

The two wings 44 of puncture cannula 36 should have sufficient flexibility to be able to lie adjacent to the cross-section of arterial flexible-tube line 6 with clamped fixing thereof. It is however also possible to design the wings as slightly arc-shaped in order to facilitate the placing onto the flexible-tube line. In this case, the wings can have a lesser flexibility.

If the puncture cannula is joined with the flexible-tube line, the axes of the flexible-tube lines and cannulas again run parallel to one another. Pulling-out of the venous cannula again leads to slipping-out of the arterial cannula, as a result of which an alarm is triggered.

1-14. (canceled)

15. A device for monitoring vascular access in extracorporeal blood treatment comprising:
a first attachment element adapted to removably receive a flexible arterial tube or an arterial cannula,
a second attachment element adapted to removably receive a flexible venous tube or a venous cannula,
whereby the arterial cannula and venous cannula are thereby held in a parallel orientation.

16. The device of claim 15, wherein the first attachment element and the second attachment element are unitary.

17. The device of claim 15, wherein the first attachment element has a substantially C-shape comprising a curved central portion and two end pieces defining a gap therebetween for receiving the arterial tube or arterial cannula.

18. The device of claim 15, wherein the second attachment element has a substantially C-shape comprising a curved central portion and two end pieces defining a gap therebetween for receiving the venous tube or venous cannula.

19. The device of claim 15, wherein the first attachment element and the second attachment element are separable.

20. The device of claim 19, wherein the first attachment element and the second attachment element can be joined together by a snap-in fastener.

21. The device of claim 20, wherein the snap-in fastener is a press-stud.

22. The device of claim 15, wherein at least one of the first attachment element or the second attachment element has a central portion and a first wing and second wing extending therefrom, said central portion having a tubular opening adapted to receive one of the arterial tube, arterial cannula, venous tube or venous cannula.

23. The device of claim 22, wherein the first wing and second wing are flexible and are adapted to rotate around a central axis of the tubular opening.

24. The device of claim 23, wherein the first wing has a first free end and the second wing has a second free end, and the first free end and the second free end are adapted to be joined together to create an eyelet to receive one of the venous tube, venous cannula, arterial tube, or arterial cannula.

25. The device of claim 24, wherein the first free end and the second free end can be joined together by a snap-in fastener.

26. The device of claim 25, wherein the snap-in fastener is a press-stud.

27. The device of claim 15, wherein the first attachment element and the second attachment element are designed in such a way that the arterial cannula is pulled out if the venous cannula slips out.

28. A method for monitoring a vascular access to a patient in an extracorporeal blood treatment, comprising:
removing blood from the patient via a flexible arterial tube that has an arterial cannula;
returning blood to the patient via a flexible venous tube that has a venous cannula;
separably joining the arterial cannula or the arterial tube with the venous cannula or venous tube in an essentially parallel orientation, so that the arterial cannula is pulled out in the event of the venous cannula becoming detached; and
monitoring the pressure and/or air intake in the arterial line, wherein it is determined that there is an incorrect patient access if there is a change in the pressure and/or air intake outside preset limits.
